(19) United States
(12) Patent Application Publication
(10) Pub. No.: US 2025/0195089 A1
(43) Pub. Date: Jun. 19, 2025

(54) BLOOD LOSS SYSTEMS, METHODS, AND DEVICES

(71) Applicant: Walk Vascular, LLC, Irvine, CA (US)

(72) Inventors: Matthew J. Gillick, Murrieta, CA (US); Puneet Kamal Singh Gill, Anaheim, CA (US); Stephanie Anne Maldonado, Winchester, CA (US)

(21) Appl. No.: 18/950,518

(22) Filed: Nov. 18, 2024

Related U.S. Application Data
(60) Provisional application No. 63/609,442, filed on Dec. 13, 2023.

(51) Int. Cl. A61B 17/22; A61B 17/00

(52) U.S. Cl. CPC A61B 17/22; A61B 17/00234; A61B 2017/00238; A61B 2017/00305; A61B 2017/00561; A61B 2017/22079; A61B 2217/005

(57) ABSTRACT

A system for reducing blood loss during removal of material from a body lumen includes an elongate shaft, an aspiration lumen, and a flow resistance assembly. The elongate shaft can be placed within a blood vessel and has a distal end with an opening at the distal end. The aspiration lumen extends along the shaft and has a proximal end and a distal opening. The flow resistance assembly is associated with the aspiration lumen and is configured to resist blood flow through the aspiration lumen in a free-flow condition without operating a valve in fluid communication with the aspiration lumen. The flow resistance assembly may include a coiled section of tubing, a static mixer, or flow resistance structures.

### CROSS-REFERENCE TO RELATED APPLICATIONS

This application claims priority to and the benefit of U.S. Provisional Patent Application Ser. No. 63/609,442, filed Dec. 13, 2023, and entitled “BLOOD LOSS SYSTEMS, METHODS, AND DEVICES,” the disclosure of which is incorporated herein by this reference.

### BACKGROUND

### Technical Field

The present disclosure pertains generally to medical devices and methods of their use. More particularly, the present invention pertains to aspiration and thrombectomy devices and methods of use thereof.

### Description of the Related Art

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure to macerate the thrombus and create a venturi effect to flush the macerated material away.

All of the devices described above have limitations as a result of individual design characteristics. For example, while performing clot aspiration, a thrombectomy catheter will also remove blood from the patient. Excessive blood loss is a major concern during this type of procedure, as once the blood loss volume exceeds a threshold of concern, such as approx. 0.5 to 1.0 liters, the procedure will need to be halted to protect patient safety.

When a thrombectomy catheter is engaged with a blood clot, the blood aspiration rate is very low since the clot will partially or completely occlude the catheter distal opening. When the catheter becomes disengaged from the clot or clot removal is completed, the blood aspiration rate can rise sharply and thus presents a patient safety concern.

As such, what is needed are systems, methods, and devices, that reduce the free-flow blood aspiration rate while reducing the impact on the systems and device's ability to remove blood clots, thrombus and other aspirants.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Embodiments of the present invention provide systems, methods, and devices for aspirating thrombus or material within a patient's body.

Implementations of the present invention solve one or more problems in the art with systems, methods, and devices for reducing blood loss during removal of material from a body lumen. In one implementation, a system for reducing blood loss during removal of material from a body lumen includes an elongate shaft, an aspiration lumen, and a flow resistance assembly. The elongate shaft can be placed within a blood vessel and has a distal end with an opening at the distal end. The aspiration lumen extends along the shaft and has a proximal end and a distal opening. The flow resistance assembly is associated with the aspiration lumen and is configured to resist blood flow through the aspiration lumen in a free-flow condition without operating a valve in fluid communication with the aspiration lumen.

In another configuration, a system for reducing blood loss during removal of material from a body lumen includes an elongate shaft, a supply lumen and an aspiration lumen, an orifice, and a flow resistance assembly. The elongate shaft may be configured for placement within a blood vessel and may have a distal end with an opening at the distal end. The supply lumen and the aspiration lumen each extend along the shaft. The supply lumen has a proximal end and a distal end and the aspiration lumen has a proximal end and a distal opening. The orifice is near the distal end of the supply lumen and is configured to allow injection of pressurized fluid into the aspiration lumen at or near the distal end of the aspiration lumen when the pressurized fluid is delivered through the supply lumen. The flow resistance assembly is associated with the aspiration lumen and is configured to resist blood flow through the aspiration lumen in a free-flow condition without operating a valve to at least partially compress an aspiration tube with the aspiration lumen.

In another configuration, a system for reducing blood loss during removal of material from a body lumen includes an elongate shaft, a supply lumen and an aspiration lumen, an orifice, and a flow resistance assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is near the distal end of the supply lumen and is configured to allow injection of pressurized fluid into the aspiration lumen at or near the distal end of the aspiration lumen when the pressurized fluid is delivered through the supply lumen. The flow resistance assembly is associated with the aspiration lumen and is configured to resist blood flow through the aspiration lumen in a free-flow condition with a single flow path extending from the distal end of the elongate shaft to a proximal end of the resistance flow assembly before and during aspiration of material from the body lumen.

In another configuration, a system for reducing blood loss during removal of material from a body lumen includes an elongate shaft, a supply lumen and an aspiration lumen, an orifice, and a flow resistance assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is near the distal end of the supply lumen and is configured to allow injection of pressurized fluid into the aspiration lumen at or near the distal end of the aspiration lumen when the pressurized fluid is delivered through the supply lumen. The flow resistance assembly is associated with the aspiration lumen and is configured to resist blood flow through the aspiration lumen in a free-flow condition without operating a valve in fluid communication with the aspiration lumen.

In another configuration, a system for reducing blood loss during removal of material from a body lumen includes an elongate shaft, a supply lumen and an aspiration lumen, an orifice, and a flow resistance assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is near the distal end of the supply lumen and is configured to allow injection of pressurized fluid into the aspiration lumen at or near the distal end of the aspiration lumen when the pressurized fluid is delivered through the supply lumen. The flow resistance assembly is associated with the aspiration lumen and is configured to resist blood flow through the aspiration lumen in a free-flow condition without operating a valve to at least partially compress an aspiration tube with the aspiration lumen.

In another configuration, a system for reducing blood loss during removal of material from a body lumen includes an elongate shaft, a supply lumen and an aspiration lumen, an orifice, and a flow resistance assembly. The elongate shaft is configured for placement within a blood vessel and has a distal end with an opening at the distal end. The supply lumen and the aspiration lumen each extend along the shaft. The supply lumen has a proximal end and a distal end and the aspiration lumen has a proximal end and a distal opening. The orifice is near the distal end of the supply lumen and is configured to allow injection of pressurized fluid into the aspiration lumen at or near the distal end of the aspiration lumen when the pressurized fluid is delivered through the supply lumen. The flow resistance assembly is associated with the aspiration lumen and is configured to resist blood flow through the aspiration lumen in a free-flow condition with a single flow path extending from the distal end of the elongate shaft to a proximal end of the resistance flow assembly before and during aspiration of material from the body lumen.

In another configuration, a system for aspirating thrombus includes an aspiration catheter, a tubing set, a pressurization element, and a flow resistance assembly. The aspiration catheter includes an elongate shaft, a supply lumen and an aspiration lumen, and an orifice. The elongate shaft is configured for placement within a blood vessel of a subject. The supply lumen and the aspiration lumen each extend along an interior of the elongate shaft. The shaft lumen is disposed within a supply tube disposed within a plurality of mounts disposed within the aspiration lumen. The plurality of mounts are configured to position the supply lumen towards one side of the aspiration lumen to prevent blockage of the aspiration lumen through entanglement of thrombus with the supply tube. The orifice is at or near a distal end of the supply lumen and is configured to allow injection of pressurized fluid into the aspiration lumen at or near the distal end of the aspiration lumen when the pressurized fluid is caused or allowed to flow through the supply lumen. The tubing set includes a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source. The pressurization element is configured to couple to the tubing set and is further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen. The flow resistance assembly is in fluid communication with at least one of the aspiration catheter and the tubing set and is configured to resist blood flow through at least one of the aspiration catheter and the tubing set in a free-flow condition without operating a valve in fluid communication with at least one of the aspiration catheter and the tubing set.

In another configuration, a system for aspirating thrombus includes an aspiration catheter, a tubing set, a pressurization element, and a flow resistance assembly. The aspiration catheter includes an elongate shaft, a supply lumen and an aspiration lumen, and an orifice. The elongate shaft is configured for placement within a blood vessel of a subject. The supply lumen and the aspiration lumen each extend along an interior of the elongate shaft. The shaft lumen is disposed within a supply tube disposed within a plurality of mounts disposed within the aspiration lumen. The plurality of mounts are configured to position the supply lumen towards one side of the aspiration lumen to prevent blockage of the aspiration lumen through entanglement of thrombus with the supply tube. The orifice is at or near a distal end of the supply lumen and is configured to allow injection of pressurized fluid into the aspiration lumen at or near the distal end of the aspiration lumen when the pressurized fluid is caused or allowed to flow through the supply lumen. The tubing set includes a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source. The pressurization element is configured to couple to the tubing set and is further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen. The flow resistance assembly is in fluid communication with at least one of the aspiration catheter and the tubing set and is configured to resist blood flow through at least one of the aspiration catheter and the tubing set in a free-flow condition without operating a valve to at least partially compress at least one of the aspiration catheter and the tubing set.

In another configuration, a system for aspirating thrombus includes an aspiration catheter, a tubing set, a pressurization element, and a flow resistance assembly. The aspiration catheter includes an elongate shaft, a supply lumen and an aspiration lumen, and an orifice. The elongate shaft is configured for placement within a blood vessel of a subject. The supply lumen and the aspiration lumen each extend along an interior of the elongate shaft. The shaft lumen is disposed within a supply tube disposed within a plurality of mounts disposed within the aspiration lumen. The plurality of mounts are configured to position the supply lumen towards one side of the aspiration lumen to prevent blockage of the aspiration lumen through entanglement of thrombus with the supply tube. The orifice is at or near a distal end of the supply lumen and is configured to allow injection of pressurized fluid into the aspiration lumen at or near the distal end of the aspiration lumen when the pressurized fluid is caused or allowed to flow through the supply lumen. The tubing set includes a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source. The pressurization element is configured to couple to the tubing set and is further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen. The flow resistance assembly is in fluid communication with at least one of the aspiration catheter and the tubing set and is configured to resist blood flow through at least one of the aspiration catheter and the tubing set in a free-flow condition with a single flow path extending from the distal end of the elongate shaft to a proximal end of the resistance flow assembly before and during aspiration of material from the body lumen.

In another configuration, a method of reducing blood loss during removal of a material from a body lumen includes positioning a distal end of an elongate shaft of an aspiration system within a body lumen to position an opening at the distal end toward material to be removed from a body lumen, the aspiration lumen having a proximal end and a distal opening. The method also includes actuating a vacuum source in fluid communication with the aspiration lumen to remove the material at a first flow rate, wherein when a change from the first flow rate to a second flow rate higher than the first flow rate occurs, automatically resists blood flow through the aspiration lumen without operating a valve in fluid communication.

In another configuration, a method of reducing blood loss during removal of a material from a body lumen includes positioning a distal end of an elongate shaft of an aspiration system within a body lumen to position an opening at the distal end toward material to be removed from a body lumen, the aspiration lumen having a proximal end and a distal opening. The method also includes actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen without operating a valve to at least partially compress at least one of the aspiration catheter and the tubing set.

In another configuration, a method of reducing blood loss during removal of a material from a body lumen includes positioning a distal end of an elongate shaft of an aspiration system within a body lumen to position an opening at the distal end toward material to be removed from a body lumen, the aspiration lumen having a proximal end and a distal opening. The method also includes actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen with a single flow path extending from the distal end of the elongate shaft to a proximal end of the resistance flow assembly before and during aspiration of material from the body lumen.

In another configuration, a method of reducing blood loss during removal of a material from a body lumen includes positioning a distal end of an elongate shaft of an aspiration system within a body lumen to position an opening at the distal end toward material to be removed from a body lumen, a supply lumen and an aspiration lumen each extending along the elongate shaft, the supply lumen having a proximal end and a distal end, and the aspiration lumen having a proximal end and a distal opening, and an orifice near the distal end of the supply lumen, the orifice being configured to allow injection of pressurized fluid into the aspiration lumen at or near the distal end of the aspiration lumen when the pressurized fluid is delivered through the supply lumen. The method also includes actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen without operating a valve in fluid communication with the aspiration lumen.

In another configuration, a method of reducing blood loss during removal of a material from a body lumen includes positioning a distal end of an elongate shaft of an aspiration system within a body lumen to position an opening at the distal end toward material to be removed from a body lumen, a supply lumen and an aspiration lumen each extending along the elongate shaft, the supply lumen having a proximal end and a distal end, and the aspiration lumen having a proximal end and a distal opening, and an orifice near the distal end of the supply lumen, the orifice being configured to allow injection of pressurized fluid into the aspiration lumen at or near the distal end of the aspiration lumen when the pressurized fluid is delivered through the supply lumen. The method also includes actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen without operating a valve to at least partially compress at least one of the aspiration catheter and the tubing set.

In another configuration, a method of reducing blood loss during removal of a material from a body lumen includes positioning a distal end of an elongate shaft of an aspiration system within a body lumen to position an opening at the distal end toward material to be removed from a body lumen, a supply lumen and an aspiration lumen each extending along the elongate shaft, the supply lumen having a proximal end and a distal end, and the aspiration lumen having a proximal end and a distal opening, and an orifice near the distal end of the supply lumen, the orifice being configured to allow injection of pressurized fluid into the aspiration lumen at or near the distal end of the aspiration lumen when the pressurized fluid is delivered through the supply lumen. The method also includes actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen with a single flow path extending from the distal end of the elongate shaft to a proximal end of the resistance flow assembly before and during aspiration of material from the body lumen.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a plan view of exemplary disposable components of a system for aspirating thrombus according to an implementation of the present disclosure.

FIG. 2 is a sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a plan view of exemplary disposable components of a system for aspirating thrombus according to an implementation of the present disclosure.

FIG. 5 is a perspective view of an exemplary system for aspirating thrombus of FIG. 3 according to an implementation of the present disclosure.

FIG. 6 is a perspective view of an exemplary aspiration catheter according to an implementation of the present disclosure.

FIG. 7 is a sectional view of an exemplary aspiration catheter according to an implementation of the present disclosure.

FIG. 8 illustrates the exemplary aspiration catheter within a body lumen aspirating thrombus according to an implementation of the present disclosure.

FIG. 9 illustrates an exemplary vacuum canister with a flow resistance assembly according to an implementation of the present disclosure.

FIG. 10 illustrates an exemplary tubing set with a flow resistance assembly according to an implementation of the present disclosure.

FIG. 11 illustrates an exemplary tube with a flow resistance assembly according to an implementation of the present disclosure.

FIG. 12 illustrates an exemplary flow resistance assembly according to an implementation of the present disclosure.

FIG. 13 illustrates a cross-sectional view of an exemplary tube with a flow resistance assembly according to an implementation of the present disclosure.

FIG. 14 illustrates a cross-sectional view of an exemplary tube with a flow resistance assembly according to an implementation of the present disclosure.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to reducing the blood flow rate in a free flow condition without significantly impacting the ability of the system to remove thrombus. The blood flow rate may be reduced using a flow resistance assembly, device, or feature, such as tubing with a coiled section, a static mixer, or flow resistance structures.

While the present disclosure will describe particular implementations of flow resistance assemblies, devices, and features, it should be understood that the assemblies, devices, features, systems, and methods described herein may be applicable to other uses. Additionally, elements described in relation to any embodiment depicted and/or described herein may be combinable with elements described in relation to any other embodiment depicted and/or described herein.

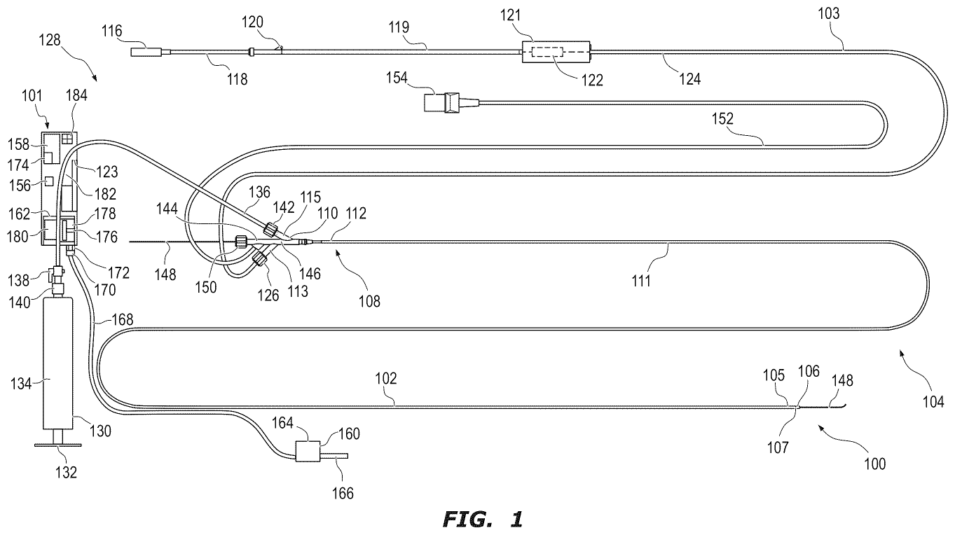

A system 100 for aspirating thrombus is illustrated in FIGS. 1-5. The system 100 for aspirating thrombus includes a pump 101, an aspiration catheter 102, and a tubing set 103. The aspiration catheter 102 and the tubing set 103 represent disposable components, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

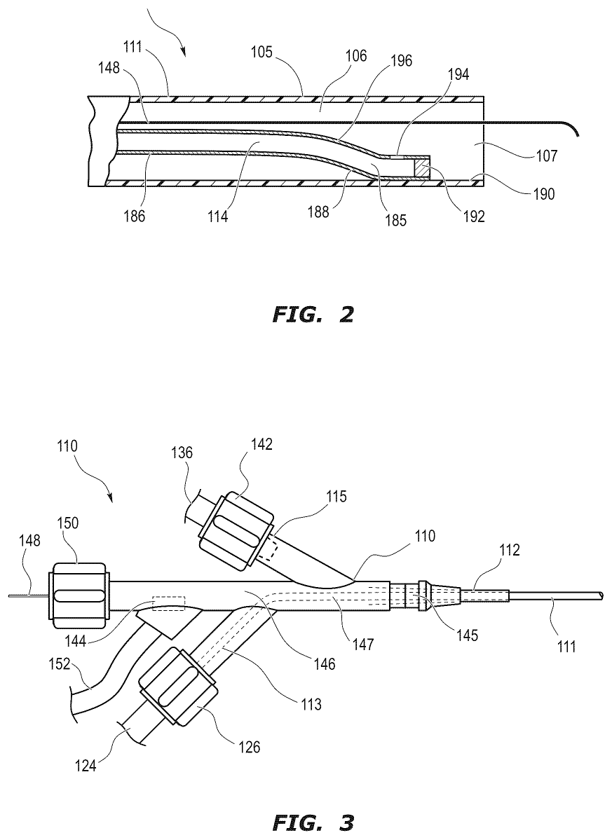

The aspiration catheter 102 has a distal end 105 and includes an over-the-wire guidewire lumen/aspiration lumen 106 extending between an open distal end 107 and a proximal end 108 comprising a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106.

A spike 116 for coupling to a fluid source (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

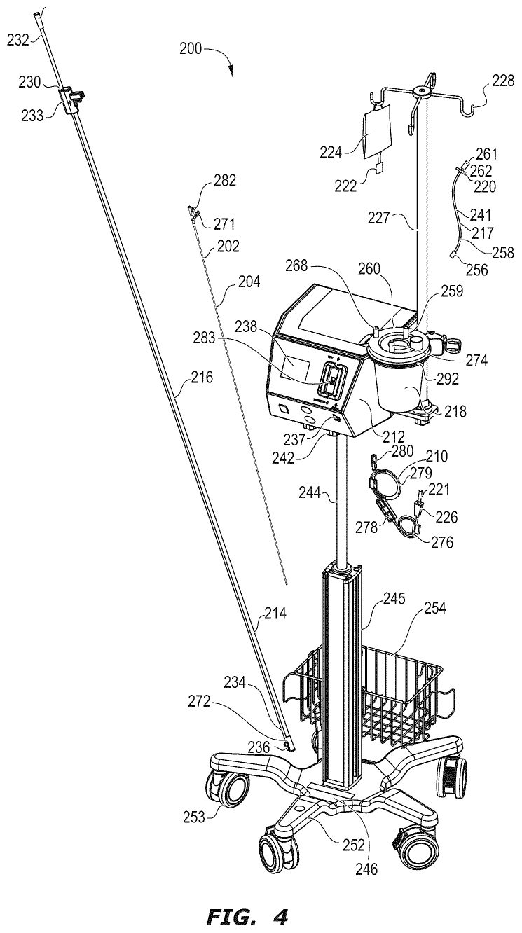

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure sensor or transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump 101 may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor or transducer 144 thus senses a negative pressure and sends a signal, causing the controller to start the motor 182 of the pump 101. As the effect via the electronics is substantially immediate, the motor 182 starts pumping almost immediately after the pedal 166 is depressed. As the pedal 166 of the foot pedal 160 is released, the controller 174 then causes the pinch valve 162 to close. The pressure sensor or transducer 144 thus senses that no negative pressure is present and the controller 174 causes the motor 182 of the pump 101 to shut off. Again, the effect via the electronics is substantially immediate, and thus the motor 182 stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torquability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

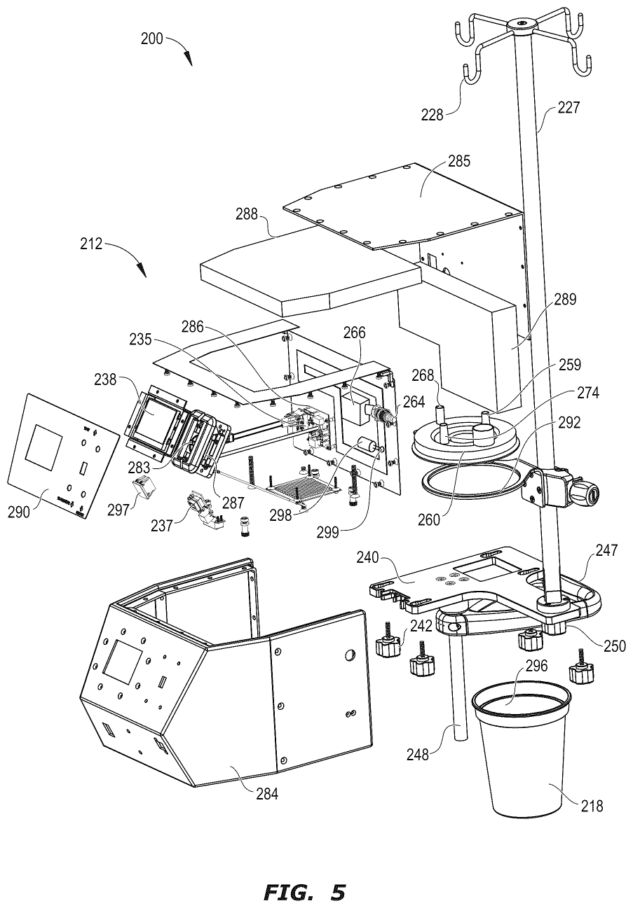

A system 200 for aspirating thrombus is illustrated in FIGS. 4-5. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIG. 3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice or opening 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114. In some embodiments, the orifice or opening 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 4 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 121 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

FIG. 5 illustrates aspects pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 268 and second port 259 for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 268 for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259 for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259 so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

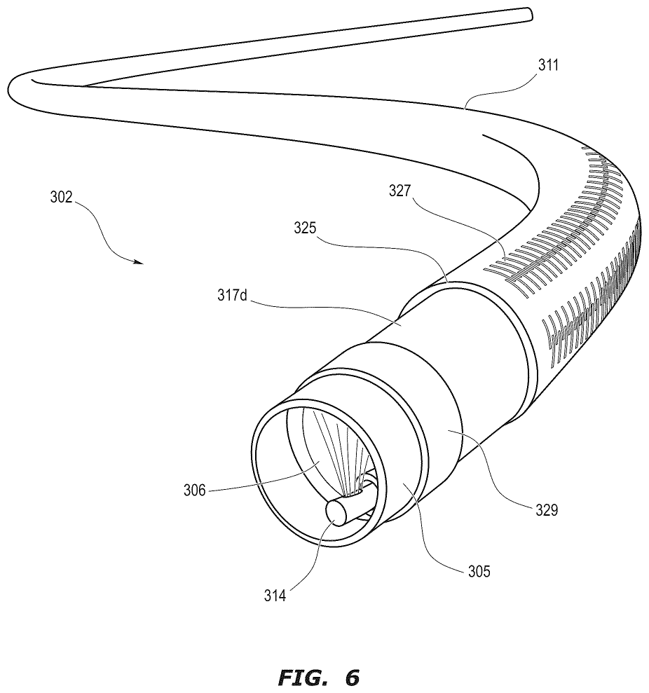

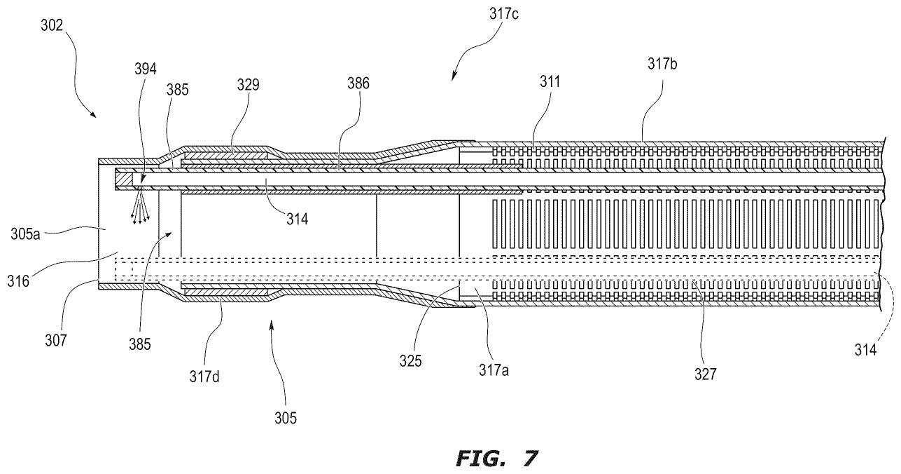

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacket by a jacket 317b. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps the distal end 325 of the shaft 311, and forms the aspiration catheter distal end 305a or the distal most end of the aspiration catheter with the distal opening 307. The outer jacket 317d protects a distal portion 385 of a supply tube 386 containing a supply lumen 314. While reference is made of a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structures to form the shaft.

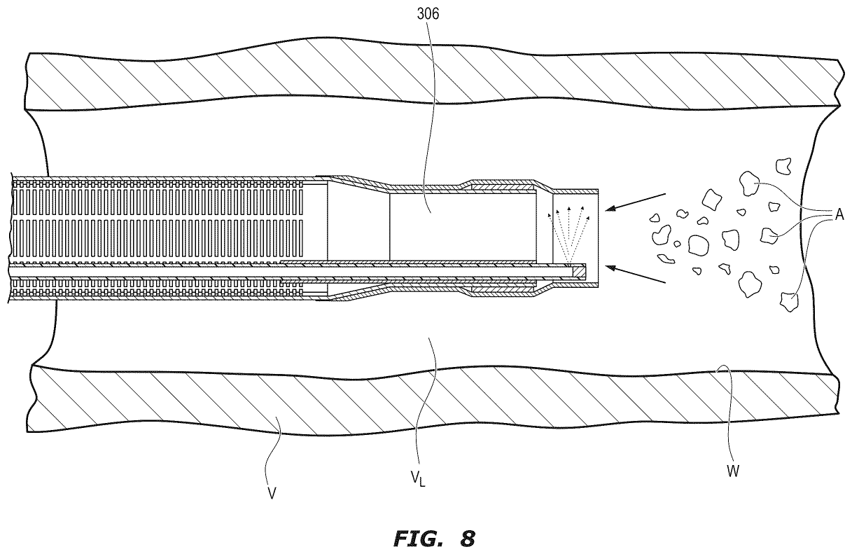

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen VL of a vessel V and aspirant A is being drawing into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, laminate or composite materials, bio-mimicking materials, and combinations or modifications thereof.

In some situations, during aspiration of thrombus, the aspiration catheter may also remove blood from the patient. Excessive blood loss can be a concern, as once the blood loss volume exceeds a threshold of concern, the procedure will need to be halted to protect the patient's safety.

When the aspiration catheter is engaged with a thrombus, the blood aspiration rate is very low since the thrombus will partially or completely occlude the catheter distal opening. When the catheter becomes disengaged from the thrombus or thrombus removal is completed, the blood aspiration rate may rise sharply and present a patient safety concern.

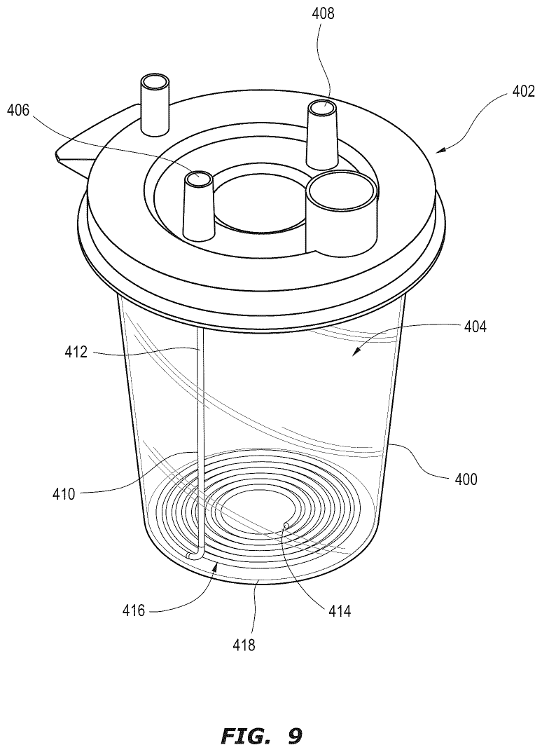

FIGS. 9-16 relate to various flow resistance assemblies, devices, features, and methods to significantly reduce the free-flow blood aspiration rate (where a thrombus is not engaged with the distal end of the aspiration catheter), while not significantly impacting the ability of the system to remove a thrombus. In some embodiments, the resistance assemblies, devices, and features may be configured to resist blood flow through the aspiration catheter/lumen and/or an associated tubing set in a free-flow condition (e.g., absent a thrombus that entirely or significantly block the aspiration lumen) without operating a valve in fluid communication with the aspiration catheter/lumen and/or associated tubing set or at least partially compressing the aspiration catheter/lumen and/or associated tubing set. In other embodiments, the resistance assemblies, devices, and features may be configured to resist blood flow through the aspiration lumen in a free-flow condition with a single flow path extending from the distal end of the aspiration lumen to a proximal end of the resistance flow assembly, device, or feature before and during aspiration of material from the body lumen. In still other embodiments, the resistance assemblies, devices, and features may be configured to resist blood flow through at least one of the aspiration catheter and the tubing set in a free-flow condition with a single flow path extending from the distal end of the elongate shaft to a proximal end of the resistance flow assembly, device, or feature before and during aspiration of material from the body lumen.

The blood aspiration flow rate is a function of two main variables: the pressure differential (ΔP) between the distal tip of the aspiration catheter and the vacuum canister, and the flow resistance of the catheter/suction tubing lumen. Since the ΔP variable will ideally be held constant, the flow resistance of the catheter/suction tubing lumen can be increased to reduce the blood aspiration rate in a free-flow condition.

The blood flow rate through the catheter/suction tubing lumen can be varied in inverse proportion to the tubing length. While increasing the flow resistance by lengthening the flow lumen will reduce the blood flow rate, it will not negatively impact thrombus removal. For instance, in a static condition, where the thrombus occludes the catheter tip, there is no flow present and therefore no flow losses. As a result, the thrombus will experience full ΔP pressure differential to pull the thrombus into to the catheter for maceration. In an active thrombus removal condition, where the thrombus is being macerated and removed, the aspiration flow rate will be very low due to partial occlusion of the catheter lumen. Accordingly, the increased flow losses of the longer lumen will have minimal impact on the aspiration rate and ΔP experienced by the thrombus. Once thrombus removal is complete (blood free-flow condition), the aspiration rate will be lowered due to the increased flow resistance of the longer lumen. This is a desirable condition since the patient blood loss rate will be reduced.

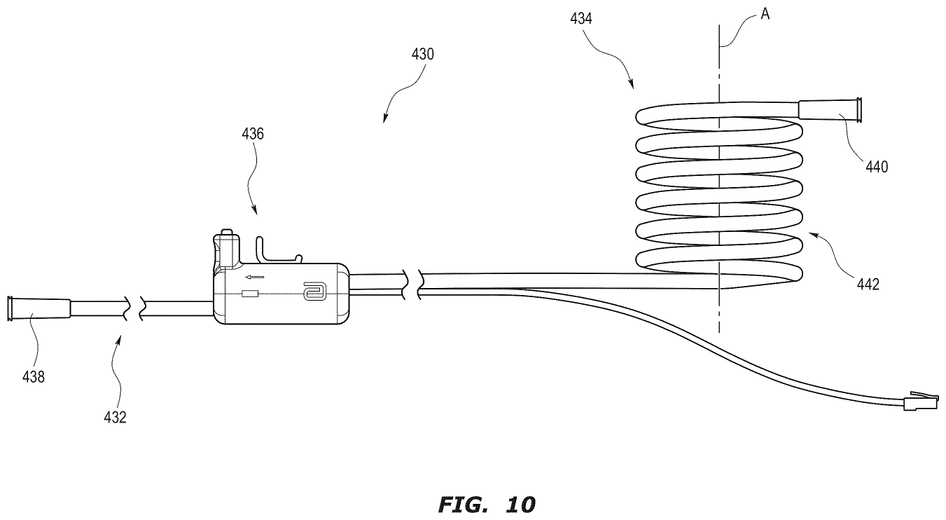

FIGS. 9 and 10 illustrate example embodiments of flow resistance assemblies, devices, and features that employ longer lumen lengths to automatically reduce the aspiration blood flow rate without operating a valve or at least partially compressing the aspiration catheter/lumen and/or associated tubing set. The example embodiments of FIGS. 9 and 10 may also be configured to automatically reduce the aspiration blood flow rate while employing a single flow path extending from the distal end of the aspiration lumen to a proximal end of the resistance flow assembly, device, or feature before and during aspiration of material from the body lumen.

FIG. 9 illustrates a vacuum canister 400 that may be similar or identical in many respects to vacuum canister 218. The canister 400 may be configured to receive and collect aspirant (e.g., thrombus, blood, saline) that is evacuated from the patient through the aspiration lumen 106. A lid 402 is configured to cover a portion of the canister 400 and close an interior 404 of the canister 400. The lid 402 may be connected to the canister 400 via a snapping, screwing, clipping, friction fitting, or other connection modality.

The lid 402 may comprise two or more ports, including a first port 406 and a second port 408 for providing negative pressure/vacuum to the aspiration lumen 106. For example, sterile suction tubing 216 may be connected to the lid 402 of the vacuum canister 400 at the first port 406 for transmitting a negative pressure to the sterile suction tubing 216 and to the aspiration lumen 106 of the aspiration catheter 102. Non-sterile suction tubing 217 may be connected to the lid 402 of the vacuum canister 400 at the second port 408 for providing a negative pressure to the vacuum canister 400. A negative pressure may be provided to the non-sterile suction tubing 217 (and to sterile suction tubing 216 and the aspiration lumen 106 connected therewith) by a vacuum source (e.g., a vacuum pump or syringe).

In the illustrated embodiment, tubing 410 is disposed within the canister 400. The tubing 410 includes a lumen extending therethrough between a proximal end 412 and a distal end 414. The proximal end 412 of the tubing 410 is connected to the lid 402 and is in fluid communication with the first port 406. As a result, aspirant (e.g., thrombus, blood, saline) that is received from the sterile suction tubing 216 may pass through the first port 406 into the lumen of the tubing 410, out of an opening in the distal end 414, and into the canister 400.

Between the proximal and distal ends 412, 414 of the tubing 410 is a coiled section 416. The coiled section 416 may include at least one turn, a plurality of turns, or an number N turns where N ranges from about 2 and about 100. The coiled section 416 may have a length of about 60 cm or between about 30 cm to about 300 cm.

As in the illustrated embodiment, the turns may be arranged in a concentric spiral pattern (e.g., such that each turn has a diameter or other dimension that is different from the other turns). The turns of the coiled section 416 may lie within a single plain, such as on a bottom surface 418 of the interior 404 of the canister 400 as illustrated. In other embodiments, the coiled section 416 may include multiple coiled sections. In some cases, the multiple coiled sections may include multiple concentric spiral patterns that are stacked on top of one another.

In other embodiments, the turns of the coiled section may be stacked on top of one another and around a common central axis, similar to a coil spring. For instance, the turns of the coiled section may be positioned against or adjacent to the inner surface of the wall of the canister 400 and extend at least partially up the height of the canister 400.

The aspiration lumen 106 and the lumen through the tubing 410 may form a single flow path for the aspirant. The tubing 410 adds additional length to the flow path, which is configured to reduce the aspiration blood flow rate in a free flow condition without significantly reducing the ability to aspirate thrombus. Additionally, positioning the tubing 410, and particularly the coiled section 416 thereof within the canister 400 does not negatively affect the handling of the catheter 102.

In some embodiments, the canister 400 and/or the tubing 410 may be formed of a transparent or translucent material. This may allow an operator to monitor the amount of blood that has been collected within the tubing 410 and/or the canister 400 to ensure that the amount of blood does not exceed a predetermined threshold.

FIG. 10 another example embodiment of flow resistance assembly, device, or feature that employs a longer lumen length to automatically reduce the aspiration blood flow rate. More specifically, FIG. 10 illustrates a tubing set 430 that may be connected between the proximal end 108 of the aspiration catheter 102 and the lid (e.g., lid 260, 402) of a vacuum canister (e.g., canister 218, 400) so that the tubing set 430 may be in fluid communication with both.

The tubing set 430 includes a first tube section 432 and a second tube section 434, each with a lumen extending therethrough. The first tube section 432 and the second tube section 434 are connected together by a control 436. The distal end of the first tube section 432 includes a connector 438 that is configured to be connected to the proximal end 108 of the aspiration catheter 102 or an intermediate structure such as the y-connector 110. Similarly, the proximal end of the second tube section 434 includes a connector 440 that is configured to be connected to a port on a vacuum canister lid or an intermediate structure such as another tubing set that can be connected between the tubing set 430 and the vacuum canister lid. The connectors 438, 440 may be any suitable type of connector, including the types disclosed elsewhere herein.

The control 436 may include a valve that can be selectively opened and closed to allow for or prevent aspiration through the aspiration catheter 102/tubing set 430. Additional details regarding example embodiments of the control 436 may be found in U.S. Pat. Publ. No. 2022/0257268, published Aug. 18, 2022, and entitled, “Systems and Methods for Removal of Blood and Thrombotic Material,” which is incorporated herein by reference in its entirety.

In the illustrated embodiment, the second tube section 434 includes a coiled section 442. The coiled section 442 may include at least one turn, a plurality of turns, or an number N turns where N ranges from about 2 and about 100. The coiled section 442 may have a length of about 60 cm or between about 30 cm to about 300 cm.

In the illustrated embodiment, the coiled section 442 includes a plurality of turns having similar diameters arranged next to one another and around and along the length of a common axis A, similar to a coil spring. In other embodiments, the turns may have different diameters or may be arranged concentrically, similar to the coiled section 416 of FIG. 9.

In the illustrated embodiment, the coiled section 442 is disposed between the control 436 and the connector 436 or associated vacuum canister. According to the illustrated embodiment, the coiled section 442 is disposed adjacent to the connector 440 or closer to the connector 440 than to the control 436. In this configuration, the coiled section 442 may be hung on a hook 228 on the IV pole 227 or other location adjacent to the vacuum canister. This configuration can prevent excess tubing from being placed within the working surgical area.

The embodiments of FIGS. 9 and 10 may be simple to manufacture for relatively low cost. Additionally, both embodiments can be fine-tuned for optimal performance by varying the lengths of the tubing section and/or the coiled section and/or the diameters of the lumens therethrough. In either case, the flow resistance assemblies, devices, or features of FIGS. 9 and 10 may comprise a coiled section of tubing that is in fluid communication with an aspiration tube. The coiled section of tubing may include a coil lumen extending therethrough. In some embodiments, a ratio of the coil lumen to the aspiration lumen is from about 1:1 to about 100:1. Similarly, in some embodiments, a ratio of a length of the coil lumen to a length of the aspiration lumen alone or in combination with a lumen of a tube set connected between the aspiration catheter and a vacuum source is from about 1:1 to about 100:1.

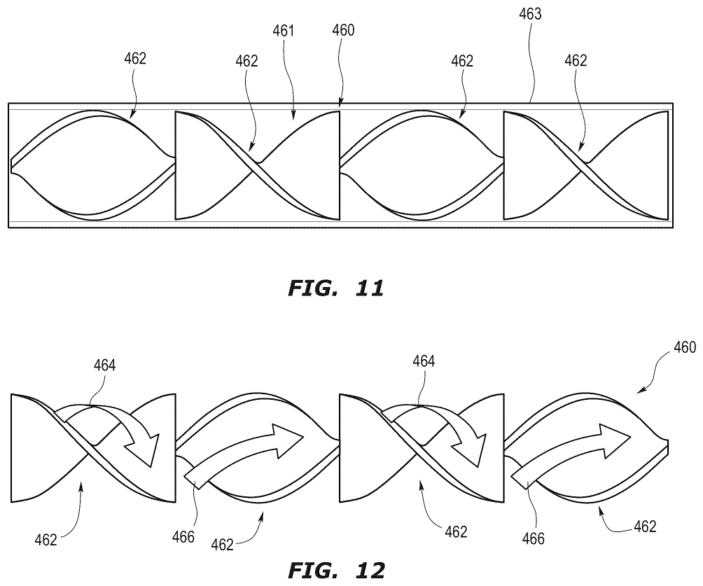

Attention is now directed to FIGS. 11 and 12, which illustrate additional embodiments of flow resistance assemblies, devices, or features that automatically reduce the aspiration blood flow rate without the use of a valve or compressing a flow lumen. The embodiments of FIGS. 11 and 12 include a flow impeding device that can be inserted into or otherwise positioned within a flow path (e.g., the aspiration lumen 106, a sterile suction tubing 136, 216, 430 extending proximally from the aspiration catheter 102, 202 toward a vacuum canister 218, 400, a non-sterile suction tubing 217 extending from the vacuum canister 218, 400 to a saline drive unit 212, a tubing 410 within a vacuum canister 400, or a portion of the aspiration lumen 434 between a control 436 and the vacuum canister 400) to create additional flow restriction and thus reduce blood loss in a free-flow condition.

One type of flow impeding device that may be employed is a static mixer 460 as illustrated in FIGS. 11 and 12. FIG. 11 illustrates the static mixer 460 disposed within a lumen 461 of a tube 463 and FIG. 12 illustrates the static mixer 462 alone. The illustrated static mixer 460 includes four helical segments 462 that are configured to direct a flow of blood and aspirant. In other embodiments, the static mixer 460 may include a plurality of helical segments, including fewer or more than four helical segments 462, or M helical segments where M ranges from about 4 and about 100.

The helical segments 462 may have similar or the same lengths as one another, or at least some of the helical segments 462 may have lengths that are different from one or more of the other helical segments 462. For instance, the helical segments 462 may have lengths of about 1 cm or ranging from about 0.2 cm to about 10 cm. The combined length of the helical segments 462 may be about 10 cm or range from about 2 cm to about 100 cm.

The helical segments 462 may have similar or the same pitch or twist as one another, or at least some of the helical segments 462 may have pitches or twists that are different from one or more of the other helical segments 462. For instance, the helical segments 462 may have a pitch or twist of about 1 turns per centimeter or ranging from about 0.5 turns per centimeter to about 5 turns per centimeter.

In the illustrated embodiment, the helical segments 462 are positioned adjacent to or in contact with one another. This is merely exemplary. In other embodiments, at least some of the helical segments 462 may be spaced apart from one another. In some embodiments, multiple static mixers 460 may be disposed at different points along the length of the flow path. The different static mixers 460 may have similar or different configurations (e.g., number of helical segments 462, lengths, etc.).

In the illustrated embodiment, at least one helical segment 462 directs the flow of blood and aspirant in a clockwise helical path as indicated by arrows 464 and at least one helical segment 462 directs the flow of blood and aspirant in a counterclockwise helical path as indicated by arrows 466. Adjacent or successive helical segments 462 may direct the flow of blood and aspirant in different helical directions. The changing helical directions of the adjacent or successive helical segments 462 can cause fluid flow to rotate around the tube axis in alternating directions. The changing direction of fluid flow can cause a significant pressure drop and flow restriction, thereby reducing the free-flow rate of blood during aspiration. In contrast, in static or low-flow conditions, such as when the aspiration catheter 102 is engaged with thrombus, there would be little to no pressure loss across the device. As a result, thrombus removal efficiency is not negatively impacted, but the blood aspiration flow rate is desirably reduced.

The embodiments of FIGS. 11 and 12 may be simple to manufacture for relatively low cost. Additionally, the static mixer 460 can be fine-tuned for optimal performance by varying the number of helical segments 462, the lengths of the helical segment 462 and/or the static mixer 460, and/or the pitch or twist of the helical segments 462.

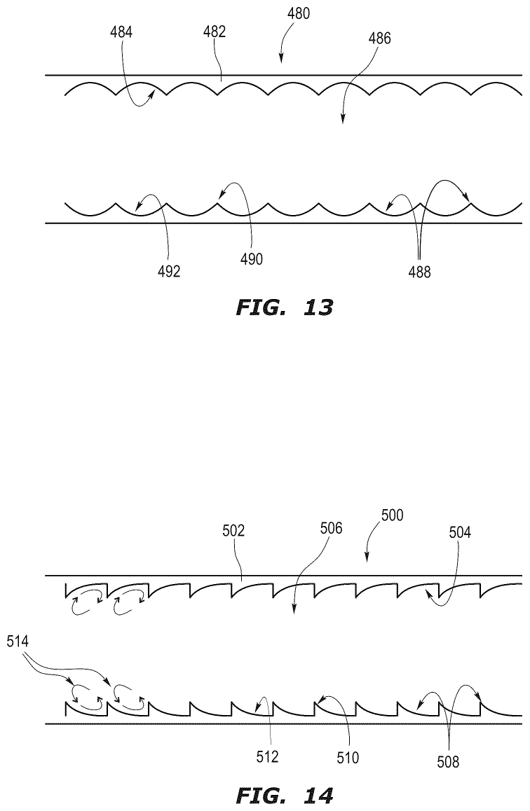

Attention is now directed to FIGS. 13-16, which illustrate additional embodiments of flow resistance assemblies, devices, or features that automatically reduce the aspiration blood flow rate without the use of a valve or compressing a flow lumen. The embodiments of FIGS. 13-16 include texturing or other modifications on the inner diameter of a flow path (e.g., the aspiration lumen 106, a sterile suction tubing 136, 216, 430 extending proximally from the aspiration catheter 102, 202 toward a vacuum canister 218, 400, a non-sterile suction tubing 217 extending from the vacuum canister 218, 400 to a saline drive unit 212, a tubing 410 within a vacuum canister 400, or a portion of the aspiration lumen 434 between a control 436 and the vacuum canister 400) to create turbulence, micro vortices, or other flow anomalies which increase resistance to flow and thus reduce blood loss in a free-flow condition. In some embodiments, the increased resistance to flow may be proportional to the fluid extraction rate.

FIG. 13 illustrates a cross-sectional view of a tube 480 that may form part of a flow path. The tube 480 has a wall 482 with an inner surface 484 that defines a lumen 486 extending therethrough. The inner surface 484 may have one or a plurality of flow resistance structures 488 formed thereon or therein. The flow resistance structure(s) 488 may individually or collectively create at least one flow anomaly to increase resistance to blood flow in the free-flow condition. The least one flow anomaly may include flow turbulence or a plurality of micro vortices. In some embodiments, the flow anomalies are in a longitudinal direction, a transverse direction, or a combination of the longitudinal direction and the transverse direction.

In FIG. 13, the flow resistance structures 488 include a series of alternating peaks 490 and valleys 492. The valleys 492 may be grooves or channels formed in the inner surface 484. In the illustrated embodiment, the peaks 490 extend circumferentially around the inner surface 484 (and perpendicular to the length of the tube 480) and radially towards the center of the lumen 486. The valleys 492 likewise extend circumferentially around the inner surface 484 (and perpendicular to the length of the tube 480), but are spaced further away from the center of the lumen 486 than the peaks 490. In the illustrated embodiment, the peaks 490 are symmetrical as are the valleys 492. The illustrated peaks 490 have a pointed configuration and the valleys 492 have a rounded configuration.

The specific configuration of the flow resistance structures 488 of FIG. 13 are merely exemplary. For instance, FIG. 14 illustrates a cross-sectional view of a tube 500 that may form part of a flow path. The tube 500 has a wall 502 with an inner surface 504 that defines a lumen 506 extending therethrough. The inner surface 504 includes one or a plurality of flow resistance structures 508 formed thereon or therein. The flow resistance structures 5088 include a series of alternating peaks 510 and valleys 512. The valleys 512 may be grooves or channels formed in the inner surface 504. In the illustrated embodiment, the peaks 510 extend circumferentially around the inner surface 504 (and perpendicular to the length of the tube 500) and radially towards the center of the lumen 506. The valleys 512 likewise extend circumferentially around the inner surface 504 (and perpendicular to the length of the tube 500), but are spaced further away from the center of the lumen 506 than the peaks 510. In the illustrated embodiment, the peaks 510 are asymmetrical. The illustrated peaks 510 have a pointed configuration with one side or face having a curved configuration and one side or face having a less curved or straight configuration. Due to the asymmetrical nature of the peaks 510, the valleys 512 are likewise asymmetrical.

As illustrated by circular arrows 514 in FIG. 14, the flow resistance structures 508 may be shaped to create turbulence, micro vortices, or other flow anomalies which increase resistance to flow and thus reduce blood loss in a free-flow condition. In some embodiments, the turbulence, micro vortices, or other flow anomalies created by the flow resistance structures 508 may help to facilitate the flow of thrombus through the center of the tube 500 while also resisting or slowing the flow of blood therethrough. For instance, the turbulence, micro vortices, or other flow anomalies may increase or at least not significantly decrease the flow rate in the center of the lumen 506 where the thrombus is flowing while also reducing the flow rate of the blood near the wall 502. Because blood is less viscous than thrombus, the blood will enter the flow resistance structures and will take longer to exit the system, thereby creating the turbulence, micro vortices, or other flow anomalies. The turbulence, micro vortices, or other flow anomalies can be designed to push thrombus in the middle of the tube/lumen based on particle size and density.

The flow resistance structures disclosed herein or any other suitable flow resistance structures may be sized for a particular application. For instance, a flow resistance structure may have a dimension that is smaller than a thrombus or blood clot size being aspirated. For instance, a flow resistance structure may have a depth (e.g., radial distance between the tip of the peak/hill and the bottom of the valley) that is smaller than the thrombus or blood clot. Similarly, a flow resistance structure may have a length (e.g., distance between successive peaks or valleys) that is smaller than the thrombus or blood clot. Still further, a flow resistance structure may have a circumferential dimension that is smaller than the thrombus or blood clot. In some embodiments, the diameter dimensions are about 500 micron or range from about 30 micron to about 4000 micron. Additionally, the length of the flow resistance structures (as measured pre-procedure) can be up to 100 cm.

It will be appreciated that the flow resistance structures may be uniform or non-uniform along all or a portion of the length of a flow pathway. For instance, the flow resistance structures may be the same, including in size and shape, along all or a portion of the flow pathway. Alternatively, the flow resistance structures may be different along at least portions of the flow pathway. By way of example, the size and/or shape of the flow resistance structures may vary or be different along different portions of the flow pathway.

The turbulence, micro vortices, or other flow anomalies created by the flow resistance structures can allow for efficient removal of thrombus while limiting the amount of blood that is withdrawn from a patient. For instance, the relatively lower viscosity blood will flow into the flow resistance structures and create the flow anomalies, which cause the blood to flow through the catheter more slowly. In contrast, due to the higher viscosity, higher mass, and larger size of the thrombus compared to the blood and the flow resistance structures, the thrombus is less likely to flow into the flow resistance structures and get caught in the flow anomalies. As a result, the thrombus will follow a straighter path through the catheter, thereby flow out of the catheter sooner than the blood.

Although the systems for aspirating thrombus described herein are predominantly focused on aspiration, the systems may also, or alternatively, be configured for injecting or infusing fluids, with or without drugs, and may incorporate related features described in U.S. Pat. No. 10,716,583, issued Jul. 21, 2020, and entitled, “Systems and Methods for Removal of Blood and Thrombotic Material” and U.S. Pat. No. 10,492,805, issued Dec. 3, 2019, and entitled, “Systems and Methods for Thrombosis and Delivery of an Agent.”

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Following are some further example embodiments of the invention. These are presented only by way of example and are not intended to limit the scope of the invention in any way. Further, any example embodiment can be combined with one or more of the example embodiments.

Embodiment 1. A system for reducing blood loss during removal of material from a body lumen, the system comprising: an elongate shaft configured for placement within a blood vessel, the elongate shaft having a distal end with an opening at the distal end; an aspiration lumen extending along the shaft, the aspiration lumen having a proximal end and a distal opening; and a flow resistance assembly associated with the aspiration lumen, the flow resistance assembly being configured to resist blood flow through the aspiration lumen in a free-flow condition without operating a valve in fluid communication with the aspiration lumen.

Embodiment 2. The system of embodiment 1, wherein the flow resistance assembly comprises a coil of tubing in fluid communication with the aspiration tube.

Embodiment 3. The system of embodiment 2, wherein the coil of tubing is disposed within a vacuum canister in fluid communication with the aspiration lumen.

Embodiment 4. The system of embodiment 2, wherein the coil of tubing is disposed between a control and a vacuum canister.

Embodiment 5. The system of embodiment 2, wherein the coil of tubing comprises a coil formed in the aspiration tube.

Embodiment 6. The system of any of embodiments 2-5, wherein the coil of tubing has a length of about 30 cm to about 300 cm.

Embodiment 7. The system of any of embodiments 2-5, wherein the coil of tubing has at least one turn.

Embodiment 8. The system of any of embodiments 2-5, wherein the coil of tubing has a plurality of turns.

Embodiment 9. The system of any of embodiments 2-5, wherein the coil of tubing has a number N turns, N ranges from about 2 and about 100.

Embodiment 10. The system of embodiment 1, wherein the flow low resistance assembly comprises a static mixer disposed within the aspiration lumen.

Embodiment 11. The system of embodiment 1, wherein the flow low resistance assembly comprises a static mixer in fluid communication with the aspiration lumen.

Embodiment 12. The system of embodiment 11, wherein the static mixer is disposed in (i) the aspiration lumen, (ii) a sterile suction tubing extending proximally from the aspiration catheter toward a vacuum canister, (iii) a non-sterile suction tubing extending from the vacuum canister to a saline drive unit, or (iv) a portion of the aspiration lumen between a control and the vacuum canister.

Embodiment 13. The system of embodiment 11, wherein the static mixer comprises a plurality of helical segments configured to direct a flow of blood and aspirant.

Embodiment 14. The system of embodiment 13, wherein at least one helical segments of the plurality of helical segments directs the flow of blood and aspirant in a clockwise helical path and at least one helical segments of the plurality of helical segments directs the flow of blood and aspirant in a counterclockwise helical path.

Embodiment 15. The system of embodiment 14, wherein adjacent helical segments direct the flow of blood and aspirant in different helical directions.

Embodiment 16. The system of embodiment 13, wherein the plurality of helical segments comprise M helical segments where M ranges from about 4 and about 100.

Embodiment 17. The system of embodiment 1, wherein the flow resistance assembly comprises a plurality of flow resistance structures formed on a wall of the aspiration catheter and/or a wall of suction tubing.

Embodiment 18. The system of embodiment 17, wherein the plurality of flow resistance structures collectively create at least one flow anomaly to increase resistance to blood flow in the free-flow condition.

Embodiment 19. The system of embodiment 18, wherein the flow the least one flow anomaly comprises flow turbulence or a plurality of micro vortices.

Embodiment 20. The system of embodiment 18, wherein the plurality of resistance structures form flow anomalies in a longitudinal direction, a transverse direction, or a combination of the longitudinal direction and the transverse direction.

Embodiment 21. The system of embodiment 17, wherein each flow resistance structure has a dimension smaller than a clot size, the dimension being selected from (i) a depth of the flow resistance structure into the wall of the aspiration catheter and/or the wall of suction tubing, (ii) a length of the flow resistance structure in a longitudinal direction of the aspiration catheter and/or the wall of suction tubing, or (iii) a circumferential dimension of the flow resistance structure extending circumferentially in the aspiration catheter and/or the wall of suction tubing.

Embodiment 22. The system of embodiment 21, wherein the diameter dimension ranges from about 30 micron to about 4000 micron.

Embodiment 23. The system of embodiment 1, further comprising a supply lumen extending along the shaft, the supply lumen having a proximal end and a distal end.

Embodiment 24. The system of embodiment 23, further comprising an orifice near the distal end of the supply lumen, the orifice being configured to allow injection of pressurized fluid into the aspiration lumen at or near the distal end of the aspiration lumen when the pressurized fluid is delivered through the supply lumen.

Embodiment 25. The system of embodiment 1, further comprising a tubing set comprising a first conduit having a distal end configured to couple to the aspiration lumen of the aspiration catheter and a proximal end configured to couple to a vacuum source.

Embodiment 26. The system of embodiment 25, further comprising a vacuum canister in fluid communication with the first conduit, the aspiration lumen and a vacuum source.

Embodiment 27. The system of embodiment 25, further comprising a second conduit having a distal end configured to couple to the supply lumen of the aspiration catheter and a proximal end configured to couple to a first fluid source.

Embodiment 28. The system of embodiment 27, further comprising a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen.

Embodiment 29. The system of embodiment 1, wherein the flow resistance assembly comprises a coil of tubing in fluid communication with the aspiration tube, the coil of tubing comprising a coil lumen, wherein a ratio of the coil lumen to the aspiration lumen is from about 1:1 to about 100:1.

Embodiment 30. The method of embodiment 1, wherein the flow resistance assembly comprises a coil of tubing in fluid communication with the aspiration tube, the coil of tubing comprising a coil lumen, wherein a ratio of a length of the coil lumen to a length of the aspiration lumen and a lumen of a tube set comprising a first conduit having a distal end configured to couple to the aspiration lumen of the aspiration catheter and a proximal end configured to couple to a vacuum source is from about 1:1 to about 100:1.

Embodiment 31. A system for reducing blood loss during removal of material from a body lumen, the system comprising: an elongate shaft configured for placement within a blood vessel, the elongate shaft having a distal end with an opening at the distal end; a supply lumen and an aspiration lumen each extending along the shaft, the supply lumen having a proximal end and a distal end, and the aspiration lumen having a proximal end and a distal opening; an orifice near the distal end of the supply lumen, the orifice being configured to allow injection of pressurized fluid into the aspiration lumen at or near the distal end of the aspiration lumen when the pressurized fluid is delivered through the supply lumen; and a flow resistance assembly associated with the aspiration lumen, the flow resistance assembly being configured to resist blood flow through the aspiration lumen in a free-flow condition without operating a valve to at least partially compress an aspiration tube with the aspiration lumen.

Embodiment 32. The system of embodiment 31, wherein the flow resistance assembly comprises a coil of tubing in fluid communication with the aspiration tube.

Embodiment 33. The system of embodiment 32, wherein the coil of tubing is disposed within a vacuum canister in fluid communication with the aspiration lumen.

Embodiment 34. The system of embodiment 32, wherein the coil of tubing is disposed between a control and a vacuum canister.

Embodiment 35. The system of embodiment 32, wherein the coil of tubing comprises a coil formed in the aspiration tube.

Embodiment 36. The system of any one of embodiments 32-35, wherein the coil of tubing has a length of about 30 cm to about 300 cm.

Embodiment 37. The system of any one of embodiments 32-35, wherein the coil of tubing has at least one turn.

Embodiment 38. The system of any one of embodiments 32-35, wherein the coil of tubing has a plurality of turns.

Embodiment 39. The system of any one of embodiments 32-35, wherein the coil of tubing has a number N turns, N ranges from about 2 and about 100.

Embodiment 40. The system of embodiment 31, wherein the flow low resistance assembly comprises a static mixer disposed within the aspiration lumen.

Embodiment 41. The system of embodiment 31, wherein the flow low resistance assembly comprises a static mixer in fluid communication with the aspiration lumen.

Embodiment 42. The system of embodiment 41, wherein the static mixer is disposed in (i) the aspiration lumen, (ii) a sterile suction tubing extending proximally from the aspiration catheter toward a vacuum canister, (iii) a non-sterile suction tubing extending from the vacuum canister to a saline drive unit, or (iv) a portion of the aspiration lumen between a control and the vacuum canister.

Embodiment 43. The system of embodiment 41, wherein the static mixer comprises a plurality of helical segments configured to direct a flow of blood and aspirant.

Embodiment 44. The system of embodiment 43, wherein at least one helical segments of the plurality of helical segments directs the flow of blood and aspirant in a clockwise helical path and at least one helical segments of the plurality of helical segments directs the flow of blood and aspirant in a counterclockwise helical path.

Embodiment 45. The system of embodiment 44, wherein adjacent helical segments direct the flow of blood and aspirant in different helical directions.

Embodiment 46. The system of embodiment 43, wherein the plurality of helical segments comprise M helical segments where M ranges from about 4 and about 100.

Embodiment 47. The system of embodiment 31, wherein the flow resistance assembly comprises a plurality of flow resistance structures formed on a wall of the aspiration catheter and/or a wall of suction tubing.

Embodiment 48. The system of embodiment 47, wherein the plurality of flow resistance structures collectively create at least one flow anomaly to increase resistance to blood flow in the free-flow condition.

Embodiment 49. The system of embodiment 48, wherein the flow the least one flow anomaly comprises flow turbulence or a plurality of micro vortices.

Embodiment 50. The system of embodiment 48, wherein the plurality of resistance structures form flow anomalies in a longitudinal direction, a transverse direction, or a combination of the longitudinal direction and the transverse direction.

Embodiment 51. The system of embodiment 47, wherein each flow resistance structure has a dimension smaller than a clot size, the dimension being selected from (i) a depth of the flow resistance structure into the wall of the aspiration catheter and/or the wall of suction tubing, (ii) a length of the flow resistance structure in a longitudinal direction of the aspiration catheter and/or the wall of suction tubing, or (iii) a circumferential dimension of the flow resistance structure extending circumferentially in the aspiration catheter and/or the wall of suction tubing.

Embodiment 52. The system of embodiment 51, wherein the diameter dimension ranges from about 30 micron to about 4000 micron.

Embodiment 53. The system of embodiment 31, further comprising a supply lumen each extending along the shaft, the supply lumen having a proximal end and a distal end.

Embodiment 54. The system of embodiment 53, further comprising an orifice near the distal end of the supply lumen, the orifice being configured to allow injection of pressurized fluid into the aspiration lumen at or near the distal end of the aspiration lumen when the pressurized fluid is delivered through the supply lumen.

Embodiment 55. The system of embodiment 31, further comprising a tubing set comprising a first conduit having a distal end configured to couple to the aspiration lumen of the aspiration catheter and a proximal end configured to couple to a vacuum source.

Embodiment 56. The system of embodiment 55, further comprising a vacuum canister in fluid communication with the first conduit, the aspiration lumen and a vacuum source.

Embodiment 57. The system of embodiment 55, further comprising a second conduit having a distal end configured to couple to the supply lumen of the aspiration catheter and a proximal end configured to couple to a first fluid source.

Embodiment 58. The system of embodiment 57, further comprising a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen.

Embodiment 59. The system of embodiment 31, wherein the flow resistance assembly comprises a coil of tubing in fluid communication with the aspiration tube, the coil of tubing comprising a coil lumen, wherein a ratio of the coil lumen to the aspiration lumen is from about 1:1 to about 100:1.

Embodiment 60. The method of embodiment 31, wherein the flow resistance assembly comprises a coil of tubing in fluid communication with the aspiration tube, the coil of tubing comprising a coil lumen, wherein a ratio of a length of the coil lumen to a length of the aspiration lumen and a lumen of a tube set comprising a first conduit having a distal end configured to couple to the aspiration lumen of the aspiration catheter and a proximal end configured to couple to a vacuum source is from about 1:1 to about 100:1.

Embodiment 61. A system for reducing blood loss during removal of material from a body lumen, the system comprising: an elongate shaft configured for placement within a blood vessel, the elongate shaft having a distal end with an opening at the distal end; a supply lumen and an aspiration lumen each extending along the shaft, the supply lumen having a proximal end and a distal end, and the aspiration lumen having a proximal end and a distal opening; an orifice near the distal end of the supply lumen, the orifice being configured to allow injection of pressurized fluid into the aspiration lumen at or near the distal end of the aspiration lumen when the pressurized fluid is delivered through the supply lumen; and a flow resistance assembly associated with the aspiration lumen, the flow resistance assembly being configured to resist blood flow through the aspiration lumen in a free-flow condition with a single flow path extending from the distal end of the elongate shaft to a proximal end of the resistance flow assembly before and during aspiration of material from the body lumen.

Embodiment 62. The system of embodiment 61, wherein the flow resistance assembly comprises a coil of tubing in fluid communication with the aspiration tube.

Embodiment 63. The system of embodiment 62, wherein the coil of tubing is disposed within a vacuum canister in fluid communication with the aspiration lumen.

Embodiment 64. The system of embodiment 62, wherein the coil of tubing is disposed between a control and a vacuum canister.

Embodiment 65. The system of embodiment 62, wherein the coil of tubing comprises a coil formed in the aspiration tube.

Embodiment 66. The system of any one of embodiments 62-65, wherein the coil of tubing has a length of about 30 cm to about 300 cm.

Embodiment 67. The system of any one of embodiments 62-65, wherein the coil of tubing has at least one turn.

Embodiment 68. The system of any one of embodiments 62-65, wherein the coil of tubing has a plurality of turns.

Embodiment 69. The system of any one of embodiments 62-65, wherein the coil of tubing has a number N turns, N ranges from about 2 and about 100.

Embodiment 70. The system of embodiment 61, wherein the flow low resistance assembly comprises a static mixer disposed within the aspiration lumen.

Embodiment 71. The system of embodiment 61, wherein the flow low resistance assembly comprises a static mixer in fluid communication with the aspiration lumen.

Embodiment 72. The system of embodiment 71, wherein the static mixer is disposed in (i) the aspiration lumen, (ii) a sterile suction tubing extending proximally from the aspiration catheter toward a vacuum canister, (iii) a non-sterile suction tubing extending from the vacuum canister to a saline drive unit, or (iv) a portion of the aspiration lumen between a control and the vacuum canister.

Embodiment 73. The system of embodiment 71, wherein the static mixer comprises a plurality of helical segments configured to direct a flow of blood and aspirant.

Embodiment 74. The system of embodiment 73, wherein at least one helical segments of the plurality of helical segments directs the flow of blood and aspirant in a clockwise helical path and at least one helical segments of the plurality of helical segments directs the flow of blood and aspirant in a counterclockwise helical path.

Embodiment 75. The system of embodiment 74, wherein adjacent helical segments direct the flow of blood and aspirant in different helical directions.

Embodiment 76. The system of embodiment 73, wherein the plurality of helical segments comprise M helical segments where M ranges from about 4 and about 100.

Embodiment 77. The system of embodiment 61, wherein the flow resistance assembly comprises a plurality of flow resistance structures formed on a wall of the aspiration catheter and/or a wall of suction tubing.

Embodiment 78. The system of embodiment 77, wherein the plurality of flow resistance structures collectively create at least one flow anomaly to increase resistance to blood flow in the free-flow condition.

Embodiment 79. The system of embodiment 78, wherein the flow the least one flow anomaly comprises flow turbulence or a plurality of micro vortices.

Embodiment 80. The system of embodiment 78, wherein the plurality of resistance structures form flow anomalies in a longitudinal direction, a transverse direction, or a combination of the longitudinal direction and the transverse direction.

Embodiment 81. The system of embodiment 77, wherein each flow resistance structure has a dimension smaller than a clot size, the dimension being selected from (i) a depth of the flow resistance structure into the wall of the aspiration catheter and/or the wall of suction tubing, (ii) a length of the flow resistance structure in a longitudinal direction of the aspiration catheter and/or the wall of suction tubing, or (iii) a circumferential dimension of the flow resistance structure extending circumferentially in the aspiration catheter and/or the wall of suction tubing.

Embodiment 82. The system of embodiment 81, wherein the diameter dimension ranges from about 30 micron to about 4000 micron.

Embodiment 83. The system of embodiment 61, further comprising a supply lumen each extending along the shaft, the supply lumen having a proximal end and a distal end.

Embodiment 84. The system of embodiment 83, further comprising an orifice near the distal end of the supply lumen, the orifice being configured to allow injection of pressurized fluid into the aspiration lumen at or near the distal end of the aspiration lumen when the pressurized fluid is delivered through the supply lumen.

Embodiment 85. The system of embodiment 61, further comprising a tubing set comprising a first conduit having a distal end configured to couple to the aspiration lumen of the aspiration catheter and a proximal end configured to couple to a vacuum source.

Embodiment 86. The system of embodiment 85, further comprising a vacuum canister in fluid communication with the first conduit, the aspiration lumen and a vacuum source.

Embodiment 87. The system of embodiment 85, further comprising a second conduit having a distal end configured to couple to the supply lumen of the aspiration catheter and a proximal end configured to couple to a first fluid source.

Embodiment 88. The system of embodiment 87, further comprising a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen.

Embodiment 89. The system of embodiment 61, wherein the flow resistance assembly comprises a coil of tubing in fluid communication with the aspiration tube, the coil of tubing comprising a coil lumen, wherein a ratio of the coil lumen to the aspiration lumen is from about 1:1 to about 100:1.

Embodiment 90. The method of embodiment 61, wherein the flow resistance assembly comprises a coil of tubing in fluid communication with the aspiration tube, the coil of tubing comprising a coil lumen, wherein a ratio of a length of the coil lumen to a length of the aspiration lumen and a lumen of a tube set comprising a first conduit having a distal end configured to couple to the aspiration lumen of the aspiration catheter and a proximal end configured to couple to a vacuum source is from about 1:1 to about 100:1.

Embodiment 91. A system for reducing blood loss during removal of material from a body lumen, the system comprising: an elongate shaft configured for placement within a blood vessel, the elongate shaft having a distal end with an opening at the distal end; a supply lumen and an aspiration lumen each extending along the shaft, the supply lumen having a proximal end and a distal end, and the aspiration lumen having a proximal end and a distal opening; an orifice near the distal end of the supply lumen, the orifice being configured to allow injection of pressurized fluid into the aspiration lumen at or near the distal end of the aspiration lumen when the pressurized fluid is delivered through the supply lumen; and a flow resistance assembly associated with the aspiration lumen, the flow resistance assembly being configured to resist blood flow through the aspiration lumen in a free-flow condition without operating a valve in fluid communication with the aspiration lumen.

Embodiment 92. A system for reducing blood loss during removal of material from a body lumen, the system comprising: an elongate shaft configured for placement within a blood vessel, the elongate shaft having a distal end with an opening at the distal end; a supply lumen and an aspiration lumen each extending along the shaft, the supply lumen having a proximal end and a distal end, and the aspiration lumen having a proximal end and a distal opening; an orifice near the distal end of the supply lumen, the orifice being configured to allow injection of pressurized fluid into the aspiration lumen at or near the distal end of the aspiration lumen when the pressurized fluid is delivered through the supply lumen; and a flow resistance assembly associated with the aspiration lumen, the flow resistance assembly being configured to resist blood flow through the aspiration lumen in a free-flow condition without operating a valve to at least partially compress an aspiration tube with the aspiration lumen.

Embodiment 93. A system for reducing blood loss during removal of material from a body lumen, the system comprising: an elongate shaft configured for placement within a blood vessel, the elongate shaft having a distal end with an opening at the distal end; a supply lumen and an aspiration lumen each extending along the shaft, the supply lumen having a proximal end and a distal end, and the aspiration lumen having a proximal end and a distal opening; an orifice near the distal end of the supply lumen, the orifice being configured to allow injection of pressurized fluid into the aspiration lumen at or near the distal end of the aspiration lumen when the pressurized fluid is delivered through the supply lumen; and a flow resistance assembly associated with the aspiration lumen, the flow resistance assembly being configured to resist blood flow through the aspiration lumen in a free-flow condition with a single flow path extending from the distal end of the elongate shaft to a proximal end of the resistance flow assembly before and during aspiration of material from the body lumen.

Embodiment 94. A system for aspirating thrombus, comprising: an aspiration catheter comprising: an elongate shaft configured for placement within a blood vessel of a subject; a supply lumen and an aspiration lumen each extending along an interior of the elongate shaft, the shaft lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen; a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source; a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen; and a flow resistance assembly in fluid communication with at least one of the aspiration catheter and the tubing set, the flow resistance assembly resisting blood flow through at least one of the aspiration catheter and the tubing set in a free-flow condition without operating a valve in fluid communication with at least one of the aspiration catheter and the tubing set.

Embodiment 95. A system for aspirating thrombus, comprising: an aspiration catheter comprising: an elongate shaft configured for placement within a blood vessel of a subject; a supply lumen and an aspiration lumen each extending along an interior of the elongate shaft, the shaft lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen; a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source; a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen; and a flow resistance assembly in fluid communication with at least one of the aspiration catheter and the tubing set, the flow resistance assembly resisting blood flow through at least one of the aspiration catheter and the tubing set in a free-flow condition without operating a valve to at least partially compress at least one of the aspiration catheter and the tubing set.

Embodiment 96. A system for aspirating thrombus, comprising: an aspiration catheter comprising: an elongate shaft configured for placement within a blood vessel of a subject; a supply lumen and an aspiration lumen each extending along an interior of the elongate shaft, the shaft lumen being disposed within a supply tube disposed within a plurality of mounts disposed within the aspiration lumen, the plurality of mounts being configured to position the supply lumen towards one side of the aspiration lumen to prevent blockage of the aspiration lumen through entanglement of thrombus with the supply tube; and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen; a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source; a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen; and a flow resistance assembly in fluid communication with at least one of the aspiration catheter and the tubing set, the flow resistance assembly resisting blood flow through at least one of the aspiration catheter and the tubing set in a free-flow condition with a single flow path extending from the distal end of the elongate shaft to a proximal end of the resistance flow assembly before and during aspiration of material from the body lumen.

Embodiment 97. A method of reducing blood loss during removal of a material from a body lumen, the method comprising: positioning a distal end of an elongate shaft of an aspiration system within a body lumen to position an opening at the distal end toward material to be removed from a body lumen, the aspiration lumen having a proximal end and a distal opening; and actuating a vacuum source in fluid communication with the aspiration lumen to remove the material at a first flow rate, wherein when a change from the first flow rate to a second flow rate higher than the first flow rate occurs, automatically resisting blood flow through the aspiration lumen without operating a valve in fluid communication.

Embodiment 98. The method of embodiment 97, wherein positioning a distal end of the elongate shaft of an aspiration system further comprises positioning a supply lumen with the aspiration lumen towards the material, the supply lumen extending along the elongate shaft, the supply lumen having a proximal end and a distal end.

Embodiment 99. The method of embodiment 98, wherein positioning a distal end of the elongate shaft of an aspiration system further comprises positioning an orifice near the distal end of the supply lumen toward the material, the orifice being configured to allow injection of pressurized fluid into the aspiration lumen at or near the distal end of the aspiration lumen when the pressurized fluid is delivered through the supply lumen.

Embodiment 100. The method of embodiment 99, further comprising actuating a pressurization element configured to couple to a tubing set fluidly coupled to the aspiration lumen and further configured to pressurize fluid from a first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen.

Embodiment 101. The method of embodiment 97, wherein automatically resisting blood flow through the aspiration lumen comprises directing blood flow through a flow resistance assembly comprises a coil of tubing in fluid communication with the aspiration tube, the coil of tubing having a length to increase resistance to blood flow during the free-flow condition.

Embodiment 102. The method of embodiment 101, wherein a ratio of a length of the coil lumen to a length of the aspiration lumen is from about 1:1 to about 100:1.

Embodiment 103. The method of embodiment 101, wherein a ratio of a length of the coil lumen to a length of the aspiration lumen and a lumen of a tube set comprising a first conduit having a distal end configured to couple to the aspiration lumen of the aspiration catheter and a proximal end configured to couple to a vacuum source is from about 1:1 to about 100:1.

Embodiment 104. The method of embodiment 97, wherein automatically resisting blood flow through the aspiration lumen comprises directing blood flow sequentially along a clockwise helical path and a counterclockwise helical path in the free-flow condition.

Embodiment 105. The method of embodiment 97, wherein automatically resisting blood flow through the aspiration lumen comprises generating a flow anomaly to increase resistance to blood flow in the free-flow condition.

Embodiment 106. The method of embodiment 105, wherein generating the flow anomaly comprises forming flow anomalies in a longitudinal direction, a transverse direction, or a combination of the longitudinal direction and the transverse direction.

Embodiment 107. The method of embodiment 105, wherein generating the flow anomaly comprises forming flow turbulence or a plurality of micro vortices.

Embodiment 108. The method of embodiment 97, actuating a pressurized fluid source to jet pressurized fluid to macerate the material within the aspiration lumen.

Embodiment 109. A method of reducing blood loss during removal of a material from a body lumen, the method comprising: positioning a distal end of an elongate shaft of an aspiration system within a body lumen to position an opening at the distal end toward material to be removed from a body lumen, the aspiration lumen having a proximal end and a distal opening; and actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen without operating a valve to at least partially compress at least one of the aspiration catheter and the tubing set.

Embodiment 110. The method of embodiment 109, wherein positioning a distal end of the elongate shaft of an aspiration system further comprises positioning a supply lumen with the aspiration lumen towards the material, the supply lumen extending along the elongate shaft, the supply lumen having a proximal end and a distal end.

Embodiment 111. The method of embodiment 110, wherein positioning a distal end of the elongate shaft of an aspiration system further comprises positioning an orifice near the distal end of the supply lumen toward the material, the orifice being configured to allow injection of pressurized fluid into the aspiration lumen at or near the distal end of the aspiration lumen when the pressurized fluid is delivered through the supply lumen.

Embodiment 112. The method of embodiment 111, further comprising actuating a pressurization element configured to couple to a tubing set fluidly coupled to the aspiration lumen and further configured to pressurize fluid from a first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen.

Embodiment 113. The method of embodiment 109, wherein automatically resisting blood flow through the aspiration lumen comprises directing blood flow through a flow resistance assembly comprises a coil of tubing in fluid communication with the aspiration tube, the coil of tubing having a length to increase resistance to blood flow during the free-flow condition.

Embodiment 114. The method of embodiment 113, wherein a ratio of a length of the coil lumen to a length of the aspiration lumen is from about 1:1 to about 100:1.

Embodiment 115. The method of embodiment 113, wherein a ratio of a length of the coil lumen to a length of the aspiration lumen and a lumen of a tube set comprising a first conduit having a distal end configured to couple to the aspiration lumen of the aspiration catheter and a proximal end configured to couple to a vacuum source is from about 1:1 to about 100:1.

Embodiment 116. The method of embodiment 109, wherein automatically resisting blood flow through the aspiration lumen comprises directing blood flow sequentially along a clockwise helical path and a counterclockwise helical path in the free-flow condition.

Embodiment 117. The method of embodiment 109, wherein automatically resisting blood flow through the aspiration lumen comprises generating a flow anomaly to increase resistance to blood flow in the free-flow condition.

Embodiment 118. The method of embodiment 117, wherein generating the flow anomaly comprises forming flow anomalies in a longitudinal direction, a transverse direction, or a combination of the longitudinal direction and the transverse direction.

Embodiment 119. The method of embodiment 117, wherein generating the flow anomaly comprises forming flow turbulence or a plurality of micro vortices.

Embodiment 120. The method of embodiment 109, actuating a pressurized fluid source to jet pressurized fluid to macerate the material within the aspiration lumen.

Embodiment 121. A method of reducing blood loss during removal of a material from a body lumen, the method comprising: positioning a distal end of an elongate shaft of an aspiration system within a body lumen to position an opening at the distal end toward material to be removed from a body lumen, the aspiration lumen having a proximal end and a distal opening; and actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resisting blood flow through the aspiration lumen with a single flow path extending from the distal end of the elongate shaft to a proximal end of the resistance flow assembly before and during aspiration of material from the body lumen.

Embodiment 122. The method of embodiment 121, wherein positioning a distal end of the elongate shaft of an aspiration system further comprises positioning a supply lumen with the aspiration lumen towards the material, the supply lumen extending along the elongate shaft, the supply lumen having a proximal end and a distal end.

Embodiment 123. The method of embodiment 122, wherein positioning a distal end of the elongate shaft of an aspiration system further comprises positioning an orifice near the distal end of the supply lumen toward the material, the orifice being configured to allow injection of pressurized fluid into the aspiration lumen at or near the distal end of the aspiration lumen when the pressurized fluid is delivered through the supply lumen.

Embodiment 124. The method of embodiment 123, further comprising actuating a pressurization element configured to couple to a tubing set fluidly coupled to the aspiration lumen and further configured to pressurize fluid from a first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen.

Embodiment 125. The method of embodiment 121, wherein automatically resisting blood flow through the aspiration lumen comprises directing blood flow through a flow resistance assembly comprises a coil of tubing in fluid communication with the aspiration tube, the coil of tubing having a length to increase resistance to blood flow during the free-flow condition.

Embodiment 126. The method of embodiment 125, wherein a ratio of a length of the coil lumen to a length of the aspiration lumen is from about 1:1 to about 100:1.

Embodiment 127. The method of embodiment 125, wherein a ratio of a length of the coil lumen to a length of the aspiration lumen and a lumen of a tube set comprising a first conduit having a distal end configured to couple to the aspiration lumen of the aspiration catheter and a proximal end configured to couple to a vacuum source is from about 1:1 to about 100:1.

Embodiment 128. The method of embodiment 121, wherein automatically resisting blood flow through the aspiration lumen comprises directing blood flow sequentially along a clockwise helical path and a counterclockwise helical path in the free-flow condition.

Embodiment 129. The method of embodiment 121, wherein automatically resisting blood flow through the aspiration lumen comprises generating a flow anomaly to increase resistance to blood flow in the free-flow condition.

Embodiment 130. The method of embodiment 129, wherein generating the flow anomaly comprises forming flow anomalies in a longitudinal direction, a transverse direction, or a combination of the longitudinal direction and the transverse direction.

Embodiment 131. The method of embodiment 129, wherein generating the flow anomaly comprises forming flow turbulence or a plurality of micro vortices.

Embodiment 132. The method of embodiment 121, actuating a pressurized fluid source to jet pressurized fluid to macerate the material within the aspiration lumen.

Embodiment 133. A method of reducing blood loss during removal of a material from a body lumen, the method comprising: positioning a distal end of an elongate shaft of an aspiration system within a body lumen to position an opening at the distal end toward material to be removed from a body lumen, a supply lumen and an aspiration lumen each extending along the elongate shaft, the supply lumen having a proximal end and a distal end, and the aspiration lumen having a proximal end and a distal opening, and an orifice near the distal end of the supply lumen, the orifice being configured to allow injection of pressurized fluid into the aspiration lumen at or near the distal end of the aspiration lumen when the pressurized fluid is delivered through the supply lumen; and actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen without operating a valve in fluid communication with the aspiration lumen.

Embodiment 134. A method of reducing blood loss during removal of a material from a body lumen, the method comprising: positioning a distal end of an elongate shaft of an aspiration system within a body lumen to position an opening at the distal end toward material to be removed from a body lumen, a supply lumen and an aspiration lumen each extending along the elongate shaft, the supply lumen having a proximal end and a distal end, and the aspiration lumen having a proximal end and a distal opening, and an orifice near the distal end of the supply lumen, the orifice being configured to allow injection of pressurized fluid into the aspiration lumen at or near the distal end of the aspiration lumen when the pressurized fluid is delivered through the supply lumen; and actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen without operating a valve to at least partially compress at least one of the aspiration catheter and the tubing set.

Embodiment 135. A method of reducing blood loss during removal of a material from a body lumen, the method comprising: positioning a distal end of an elongate shaft of an aspiration system within a body lumen to position an opening at the distal end toward material to be removed from a body lumen, a supply lumen and an aspiration lumen each extending along the elongate shaft, the supply lumen having a proximal end and a distal end, and the aspiration lumen having a proximal end and a distal opening, and an orifice near the distal end of the supply lumen, the orifice being configured to allow injection of pressurized fluid into the aspiration lumen at or near the distal end of the aspiration lumen when the pressurized fluid is delivered through the supply lumen; and actuating a vacuum source in fluid communication with the aspiration lumen to remove the material at a first flow rate, wherein upon a change from the first flow rate to a second flow rate higher than the first flow rate, a flow resistance assembly associated with the aspiration lumen automatically resists blood flow through the aspiration lumen with a single flow path extending from the distal end of the elongate shaft to a proximal end of the resistance flow assembly before and during aspiration of material from the body lumen.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A system for reducing blood loss during removal of material from a body lumen, the system comprising:
an elongate shaft configured for placement within a blood vessel, the elongate shaft having a distal end with an opening at the distal end;
an aspiration lumen extending along the shaft, the aspiration lumen having a proximal end and a distal opening; and
a flow resistance assembly associated with the aspiration lumen, the flow resistance assembly being configured to resist blood flow through the aspiration lumen in a free-flow condition without operating a valve in fluid communication with the aspiration lumen.

2. The system of claim 1, wherein the flow resistance assembly comprises a coil of tubing in fluid communication with the aspiration tube, wherein the coil of tubing:
is disposed within a vacuum canister in fluid communication with the aspiration lumen;
is disposed between a control and a vacuum canister; or
comprises a coil formed in the aspiration tube.

3. The system of claim 1, wherein the flow low resistance assembly comprises a static mixer disposed within or in fluid communication with the aspiration lumen.

4. The system of claim 3, wherein the static mixer is disposed in (i) the aspiration lumen, (ii) a sterile suction tubing extending proximally from the aspiration catheter toward a vacuum canister, (iii) a non-sterile suction tubing extending from the vacuum canister to a saline drive unit, or (iv) a portion of the aspiration lumen between a control and the vacuum canister.

5. The system of claim 3, wherein the static mixer comprises a plurality of helical segments configured to direct a flow of blood and aspirant, wherein at least one helical segments of the plurality of helical segments directs the flow of blood and aspirant in a clockwise helical path and at least one helical segments of the plurality of helical segments directs the flow of blood and aspirant in a counterclockwise helical path.

6. The system of claim 1, wherein the flow resistance assembly comprises a plurality of flow resistance structures formed on a wall of the aspiration catheter and/or a wall of suction tubing, wherein the plurality of flow resistance structures collectively create at least one flow anomaly to increase resistance to blood flow in the free-flow condition.

7. The system of claim 6, wherein the plurality of resistance structures form flow anomalies in a longitudinal direction, a transverse direction, or a combination of the longitudinal direction and the transverse direction.

8. The system of claim 1, wherein each flow resistance structure has a dimension smaller than a clot size, the dimension being selected from (i) a depth of the flow resistance structure into the wall of the aspiration catheter and/or the wall of suction tubing, (ii) a length of the flow resistance structure in a longitudinal direction of the aspiration catheter and/or the wall of suction tubing, or (iii) a circumferential dimension of the flow resistance structure extending circumferentially in the aspiration catheter and/or the wall of suction tubing.

9. The system of claim 1, wherein the flow resistance assembly comprises a coil of tubing in fluid communication with the aspiration tube, the coil of tubing comprising a coil lumen, wherein a ratio of the coil lumen to the aspiration lumen is from about 1:1 to about 100:1.

10. A system for reducing blood loss during removal of material from a body lumen, the system comprising:
an elongate shaft configured for placement within a blood vessel, the elongate shaft having a distal end with an opening at the distal end;
a supply lumen and an aspiration lumen each extending along the shaft, the supply lumen having a proximal end and a distal end, and the aspiration lumen having a proximal end and a distal opening;
an orifice near the distal end of the supply lumen, the orifice being configured to allow injection of pressurized fluid into the aspiration lumen at or near the distal end of the aspiration lumen when the pressurized fluid is delivered through the supply lumen; and
a flow resistance assembly associated with the aspiration lumen, the flow resistance assembly being configured to resist blood flow through the aspiration lumen in a free-flow condition without operating a valve to at least partially compress an aspiration tube with the aspiration lumen.

11. The system of claim 10, wherein the flow resistance assembly comprises a coil of tubing in fluid communication with the aspiration tube.

12. The system of claim 11, wherein the coil of tubing comprises a coil formed in the aspiration tube or in a vacuum canister.

13. The system of claim 10, wherein the flow resistance assembly comprises a plurality of flow resistance structures formed on a wall of the aspiration catheter and/or a wall of suction tubing, wherein the plurality of flow resistance structures collectively create at least one flow anomaly to increase resistance to blood flow in the free-flow condition.

14. A system for reducing blood loss during removal of material from a body lumen, the system comprising:
an elongate shaft configured for placement within a blood vessel, the elongate shaft having a distal end with an opening at the distal end;
a supply lumen and an aspiration lumen each extending along the shaft, the supply lumen having a proximal end and a distal end, and the aspiration lumen having a proximal end and a distal opening;
an orifice near the distal end of the supply lumen, the orifice being configured to allow injection of pressurized fluid into the aspiration lumen at or near the distal end of the aspiration lumen when the pressurized fluid is delivered through the supply lumen; and
a flow resistance assembly associated with the aspiration lumen, the flow resistance assembly being configured to resist blood flow through the aspiration lumen in a free-flow condition with a single flow path extending from the distal end of the elongate shaft to a proximal end of the resistance flow assembly before and during aspiration of material from the body lumen.

15. The system of claim 14, wherein the flow low resistance assembly comprises a static mixer disposed within the aspiration lumen.

16. The system of claim 14, wherein the flow low resistance assembly comprises a static mixer in fluid communication with the aspiration lumen.

17. The system of claim 16, wherein the static mixer is disposed in (i) the aspiration lumen, (ii) a sterile suction tubing extending proximally from the aspiration catheter toward a vacuum canister, (iii) a non-sterile suction tubing extending from the vacuum canister to a saline drive unit, or (iv) a portion of the aspiration lumen between a control and the vacuum canister.

18. The system of claim 16, wherein the static mixer comprises a plurality of helical segments configured to direct a flow of blood and aspirant.

19. The system of claim 18, wherein at least one helical segments of the plurality of helical segments directs the flow of blood and aspirant in a clockwise helical path and at least one helical segments of the plurality of helical segments directs the flow of blood and aspirant in a counterclockwise helical path.

20. The system of claim 19, wherein adjacent helical segments direct the flow of blood and aspirant in different helical directions.
